(19) United States
(12) Patent Application Publication
(10) Pub. No.: US 2004/0241087 A1
(43) Pub. Date: Dec. 2, 2004

(54) Aromatase marking

(76) Inventors: Alfred Schmidt, Hamburg (DE); Heinrich Wieland, St Peter (DE)

Correspondence Address: BURNS DOANE SWECKER & MATHIS L L P, POST OFFICE BOX 1404, ALEXANDRIA, VA, 22313-1404, US

(21) Appl. No.: 10/487,953

(22) PCT Filed: Aug. 28, 2002
(86) PCT No.: PCT/EP02/09609

(30) Foreign Application Priority Data
Aug. 28, 2001 (DE) 10141937.6

(51) Int. Cl. A61K 51/00; C07F 5/00; A61K 31/56

(52) U.S. Cl. 424/1.11; 514/177; 534/11

(57) ABSTRACT

Compounds are described which exhibit an inhibitory action towards the enzyme aromatase and which comprise at least one detectable group. Such compounds are excellently suitable for medical diagnosis and/or for therapy, particularly for tumor diseases and above all for breast cancer.

The invention relates to compounds, which are very well suitable for the application in diagnosis and if necessary, optionally in addition, for the application in the therapy of disease-relevant conditions, in particular in cases of tumor diseases.

It is known to detect tumors diagnostically through detectable markers or labels, and optionally to utilize a selective enrichment of compounds labeled with radioactive nuclides in the tumor tissue. This prior art will be explained in more detail on the example of breast cancer in the following.

In U.S. Pat. No. 6,238,644, it is started from the situation that radioactive iodine (¹²⁵I or ¹³¹I) is taken up by breast cancer cells in an enhanced manner. It was observed that when administering hormones, the uptake of iodine is increased in neoplastic cells and decreased in thyroid gland cells, whereby the selectivity is improved.

In U.S. Pat. No. 4,938,948 it is proposed to apply, for the figurative illustration (imaging) of the breast cancer, monoclonal antibodies which are labeled by NMR-detectable substances or by radioactive isotopes.

EP 700 930 A1 describes a specific peptide having high affinity towards breast cancers, wherein the peptide is modified by radioactive isotopes for diagnosis and therapy.

U.S. Pat. No. 6,096,874 proposes tamoxifen derivatives having a radioactive isotope labeling for the imaging of breast cancer, that is for diagnostic purposes.

U.S. Pat. No. 4,888,163 describes an antibody labeled with radioisotopes being directed towards estriol-3-sulfate for breast cancer imaging.

Finally, the WO 9632968 A describes compounds which are derived from a benzamide structure and which are labeled with radioisotopes. The compounds have a high affinity towards the sigma receptor, which is increasingly present on the cell surfaces of tumor cells, inter alia in breast cancer.

However, the mammography is practically still applied as the diagnostic method, which is normally followed by a biopsy for verifying and distinguishing between tumor and non-tumor. It can be assumed that, alone in Germany, several millions of mammographies are carried out per year, whereupon as a rule each 30^{th} to 50^{th} patient must be subjected to a biopsy for further clarification, for eventually arriving at a diagnosis of a breast cancer in a fraction of this group. The subsequent therapeutic approaches are usually based on drastic surgical measures as well as on the use of a chemotherapy.

The object of the invention is to extend and to improve the diagnostic and optionally the therapeutic possibilities for the detection and treatment of tumors, in particular in the case of breast cancer.

This is achieved by the provision of a compound which exhibits an inhibiting action towards the aromatase enzyme, and which at the same time comprises at least one detectable group.

The aromatase, which is inhibited by the compound according to the present invention, is a cytochrome-P450-enzyme and plays a central role in the extragonadal biosynthesis of estrogens such as estradiol, estrone and estrol. Aromatase inhibitors useable according to the present invention are known—without detectable group—in a multitude of compounds, but only in connection with therapeutic approaches for the treatment of estrogen-dependent diseases such as breast cancer. In these conventional therapeutic approaches, the therapeutic effect was based on the inhibition of the aromatase enzyme alone. The incorporation of a detectable group was not contemplated. The inventive concept differs therefrom by rendering such compounds utilizable for the diagnostic field and in particular for tumor imaging, and, if necessary, by achieving an additional therapeutic effect.

By the binding of one or several detectable groups to an aromatase inhibitor, surprising advantages are achieved by the invention. Firstly, an increased aromatase activity may represent an early occurring factor in tumor formation, which is very valuable for a desired early diagnosis of abnormal or pathological conditions. Since the estrogen supply of the tumor is prenominantly based on local synthesis, the aromatase enzyme is enriched in the tumor. In addition, the tumor tissue induces an enhanced generation of aromatase in its surroundings, so that the tumor and its surroundings are characterized by substantially more aromatase molecules than the surrounding healthy tissue. Moreover, the inventive concept enables a double combined strategy, wherein the desired strategy can be controlled by the variable selection of the detectable group. Thus, the selection of the detectable group bound to the aromatase inhibitor enables to combine the use of the compound for diagnostic purposes with the concept of radiotherapy, while at the same time a disease-inhibiting and particularly a tumor-inhibiting effect can be utilized by the aromatase inhibitor in itself.

In a further aspect, the invention further provides a compound, which exhibits an inhibiting effect towards the aromatase enzyme and which is derivatised by iodine, boron and/or a metal-chelating group or by a structural moiety containing iodine, boron or a metal-chelate. For delimitation against prior art, a specific compound which is described in EP 342 665 A1 and which represents a iodine-derivative of the biologically produced substance TAN-931 is excluded. This specific compound however is proposed only for the purpose of a therapeutic use based on the inhibitory effect of the compound itself. The documents EP 775 931 A1 and EP 265 119 A1 also disclose specific compounds having anti-aromatase effect, which may, among other broad variation possibilities, be substituted by halogen, however without specifically mentioning a iodine-derivatisation. Also there, it is merely dealt with a therapeutic approach based on the inhibitory action inherent to the compound itself.

This compound having an inhibiting action towards the aromatase, which is derivatised by iodine, boron and/or a metal-chelating group or a structural moiety containing iodine, boron or a metal-chelate, is of extraordinary high benefit, because these compounds represent non-radioactive precursors and may be easily converted, depending on the desire, at any time before, preferably immediately before the use of the compounds according to the invention for diagnostic and/or therapeutic purposes, into the corresponding compound comprising the detectable group.

The invention and its features as well as its preferred embodiments will be described in more detail in the following.

As compounds having aromatase-inhibiting action suitable for the invention, preferably those are suitably selected with which a stable and relatively long-lasting enrichment is achieved in the desired target tissue where the aromatase-enzyme is present. For this purpose, those aromatase inhibitors are particularly preferred which exhibit an irreversible inhibiting action towards the aromatase enzyme. Above all, the so-called suicide-inhibitors or -substrates, which lead to a particularly efficient and persistent stable enrichment in the desired target tissue, are enumerated among the irreversible inhibiting compounds.

Such irreversible or suicide inhibitors are to be selected particularly from the group of steroidal aromatase inhibitors, because of which this class of compounds are particularly preferably utilized in the framework of the invention.

Individual compound examples of steroidal aromatase inhibitors, to which the detectable group described below is to be bound or to be coupled for the provision of the compound according to the invention, will be set forth in the following:

4-hydroxyandrost-4-ene-3,17-dione (Formestan und Lentaron),

6-methylenandrosthra-1,4-diene-3,17-dione(Exemestan),

10-(2-propynyl)estr-4-ene-3,17-dione (MDL 18962)

7 alpha substituted androstenedione-derivatives

1,4,6-androstatriene-3,17-dione (ATD)

10-oxiran-und 10-thiiran-substituted androgens

10-propargylestr-4-ene-3,17-dione

10-propargylestr-4-ene-3,17-propionate 10-(2-propynyl)-derivative

13-retro-antiprogestine

14 alpha-hydroxy-4-androstene-3,6,17-trione (14 alpha-OHAT)

16-o 19-substituted androst-4-enes

19-(cyclopropylamino)-androst-4-ene-3,17-dione

19-(ethyldithio)-androst-4-ene-3,17-dione (ORG 30958)

19-oxiranyl- and 19-thiiranyl-steroids

19-thiomethyl- and 19-azido-androstenedione

1-methyl-androsta-1,4-diene-3,17-dione (Atamestan)

2,2-dimethyl-4-hydroxy-4-androstene-3,17-dione

3 alpha-methoxyandrost-4-ene-6,17-dione

3 beta-hydroxyandrost-4-ene-6-one-derivatives

3-deoxyandrogen-19-oxygenierderivatives of 3-oxo-17 beta-carboxamido-steroids

4-(phenylthio)-4-androstene-3,17-dione

4-(thio-substituted)-4-androstene-3,17-dione

4-acetoxy-4-androstene-3,17-dione

4-aminoandrostenedione

4-androstene-3,6,17-trione

4-hydroxyandrostenedione (4-OHA, CGP 32349)

4-methoxy-4-androstene-3,17-dione

4-oxygenated androst-5-ene-17-one and their 7-oxo-derivatives

4-thiosubstituted derivatives of 4-androstene-3,17-dione

4-thiosubstituted-4-androstene-3,17-dione-derivatives

5 alpha-dihydronorethindrone (a metabolit of norethindrone)

5 alpha-reduced C19-steroids

5 alpha-androstan-17-ones with or without a carbonyl functionality at C-3 and/or C-6

6 alpha,7 alpha-ayclopropane derivatives of androst-4-ene

6 alpha-fluorotestosterone

6 beta-propynyl-substituted steroids

6,7-aziridinylsteroid und related compounds

6-alkylanalogs of delta 1,4,6-androgens

6-alkylanalogs of delta 4,6-androgens

6-alkyl-und 6-arylandrost-4-ene-3,17-dione

6-alkylandrost-4-ene-3,17-dione of 7 alpha- and 7 beta-arylaliphatic-substituted androst-4-ene-3,17-diones

6-alkylandrosta-4,6-diene-3,17-dione and their 1,4,6-triene-analogs

6-alkyl-substituted androgens

6-phenylaliphatic-substituted C19-steroids having 1,4-diene-,

4,6-diene- or 1,4,6-triene-structure

6-bromoandrostenedione

6-hydroximinoandrostenedione

6-methylenandrosta-1,4-diene-3,17-dione (FCE 24304)

6-methylenandrosta-1,4-diene-3,17-dione (FCE 24304)

6-phenylaliphatic-substituted androst-4-ene-3,17-diones

6-substituted androst-4-ene-analogs

7 alpha-(4′-amino)phenylthio-4-androstene-3,17-dione

7 alpha-substituted androsta-1,4-diene-3,17-diones

7 alpha-substituted androstenediones

7 alpha-(4′-amino)phenylthio-4-androstene-3,17-dione

7 alpha-arylaliphatic androsta-1,4-diene-3,17-diones

7 alpha-substituted androstenediones

7 substituted 4,6-androstadiene-3,17-diones

7 substituted steroids

androst-4-ene-3,6-dione derivatives

androst-5-ene-7,17-dione 19-nor- and 5 beta, 6 beta-epoxy-derivatives

A- or B-ring-substituted derivatives of androst-4-ene-3,6,17-trione

A-ring bridged steroid

bromoacetoxy 4-androstene-3-one

delta 1,4,6-androgens

delta 4,6-androgens

epimeric 6-hydroperoxyandrostenediones

estr-4-ene-3, 17-dione (MDL 18 962),

estr-4-ene-3,6,17-triones

flavonoids

RU486

The preferred irreversible inhibition is frequently revealed, if the compound according to the invention comprises, besides the detectable group, a molecular skeleton which derives from androst-4-ene, androst-5-ene, androsta-1,4-diene- or androsta-1,4,6-triene having a -3,17-dione-, a -6,17-dione-, a -7,17-dione, a -3,6,17-trione-, or a -4,7,17,19-tetraone-group, as well as the derivatives and analogs of the mentioned base structures. As suitable derivatives of the mentioned base structures, e.g. the following derivatisation types are suitable: 4-, 14- or 19-hydroxy-, 16α-halogen (wherein halogen means fluorine, chlorine, bromine or iodine), 4-halogen-lower alkylthio- (wherein halogen means fluorine, chlorine, bromine or iodine, and lower alkyl means an alkyl group having 1 to 6 C atoms, e.g. methyl, ethyl, propyl or butyl), 17α-ethinyl-, 6-oxo-, 19-nor-, 19-oxo-, 19-acetylenyl-, 4β,5β-epoxy-, 5β,6β-epoxy- as well as any combination of the mentioned derivatisation types for the provision of corresponding derivatives and analogs.

As specific examples for suitable suicide-inhibitors or -substrates, which are suitable for the binding or coupling of the detectable group for the provision of the compound of the invention, the following base compounds can be mentioned (literature citations for the description of the respective compounds as well as their obtainment are indicated in paranthesis):

6-oxo-androstendione and 19-substituted analogs, in particular the 4β,5β-epoxy-19-oxo-derivative (M. Numazawa in Jakugaku Zasshi 118(12), p. 539-553 (1998));

androst-5-ene-7,17-dione and the 19-nor-analog (M. Numazawa and M. Tachibana in Steroids 62(7), p. 516-522 (1997)); 5β,6β-expoxy-androsta-4,7,17,9-tetraone (M. Numazawa and M. Tachibana in Biol. Pharm. Bull. 20(5), p. 490-495(1997)); 4-hydroxy-androstene-dione (M. Dowsett et al. in J. Steroid

Biochem. Mol. Biol. 56(1-6 Spec. No.), p. 145-150 (1996)); norethindrone (NET, 17α-ethinyl-19-nor-testosterone) and 5α-dihydronorethindrone (T. Yamamoto et al. in Eur. J. Endocrinol. 130(6), p. 634-640 (1994));

14α-hydroxy-4-androstene-3,6,17-trione and 4-hydroxy-androstendione (J. Kitawaki et al. in J. Steroid Biochem. Mol. Biol. 44(4-6), p. 667-670 (1993));

androst-5-ene-7,17-dione as well as their 19-hydroxy-derivates (M. Numazawa et al. in Biochem. Biophys. Res. Commun. 186(1), p. 32-39 (1992));

4-[(fluoromethyl)thio]- and 4-[(chloromethyl)thio]-androstene-dione (D. Lesuisse et al. in J. Med. Chem. 35(9), p. 1588-1597 (1992));

19-acetylenyl-androstenedione (G. T. Griffing et al. in Am. J. Med. Sci. 298(2), p. 83-88 (1989));

4-hydroxy-4-androstene-3,17-dione, 4-androstene-3,6,17-dione,

1,4,6-androstene-3,17-dione, 1,4-androstene-3,17-dione and testolactone (D. F. Covey and W. F. Hood in Cancer Res. 42 (8 Suppl.), p. 3327s-3333s (1982));

17α-ethyinyl-19-nor-testosterone (Y. Osawa et al. in Science 215(4537), p. 1249-1251 (1982));

16α-halogen-androgens (wherein halogen means fluorine, chlorine, bromine or iodine) (A. M. Brodi in Cancer Res. 42(8 Suppl.), p. 3312s-3314s (1982)).

The detectable group bound or coupled to the compound according to the invention is a labeling group which is detectable via suitable single or several physical or physico-chemical methods. The labeling group particularly is one which enables a detection even if it is present within the body of a patient. The detection then is carried out specifically by the imaging or spectroscopic methods described below in more detail. In a preferred embodiment, the detectable group concurrently has a therapeutic action.

A particularly preferred embodiment of the detectable group lies in the form of a radioactive group. In this case the compound according to the invention can be used both diagnostically via the detection of the detectable group as well as therapeutically via suitably applied radio-nuclides, which may be identical or different from the diagnostically applied radio-nuclides. The radioactive group e.g. is an isotope which is selected from the group of radioactive iodine isotopes or the radioactive metal-isotopes, e.g. ¹²³J, ¹²⁵J, ¹³¹J, ¹⁸F, ⁷⁶Br, ⁷⁷Br, ¹¹¹In, ^{99m}Tc, ⁶⁷Ga, ⁶⁸Ga, ⁹⁰Y, ¹⁸⁸Re, ⁵⁶Fe ⁵⁵Mn ²⁰¹Ta, ²¹²Pb , ²¹²Bi, ¹⁵⁷Gd, ²¹¹At.

For diagnostic uses, the radioactive isotope suitably is a gamma ray-emitting emitter, e.g. ¹²³J, ¹²⁵J, ¹⁸F ⁷⁶ Br, ⁷⁷Br, ¹¹¹In and ^{99m}Tc, whereas for therapeutic uses, particle-emitting emitters such as α- and/or particularly e-emitting isotopes, e. g. ¹³¹J ⁹⁰Y ²¹¹At, ⁷⁵Br, ⁷⁷Br, ¹²¹²Pb and ²¹²Bi, are preferably applied. The isotopes may also be both particle-emitting and non-particle-emitting emitters, e.g. α- or β-emitting and/or γ-emitting emitters.

The detectable group may also be a group which can be activated to a radioactive group. In a preferred embodiment, the detectable group comprises a boron-residue or a boron-containing structural moiety such as e.g. a boroxo-, a borate- or a boric acid ester-residue, wherein the detection and optionally the therapeutic effect can be activated by neutron irradiation, which converts boron or the boron compound in an α-emitter.

A further possible embodiment for the provision of the detectable group in the compound according to the invention consists in that the detectable group comprises a magnetically active isotope. Such a magnetically active isotope is detectable e.g. by the NMR-method. Examples of such magnetically active isotopes are: e.g. ¹³C, ¹⁷O, ²H, ⁷Li, ²³Na, ³⁵Cl und ⁸⁷Rb. In the case of using a magnetically detectable group, the compound according to the invention may be used together with contrast enhancing agents, e.g. organic chelates of gadolinum in a suitable oxidation status.

A further possible embodiment of the detectable group consists in that the detectable group is a group detectable by electron spin resonance (ESR). Such a ESR-detectable substance is e.g. a group having paramagnetic metal centers such as Va(IV), Mn(II), Cu(II), Cr(III), Gd(III), Fe(II) or Fe(III), Ti(III), as well as stable organic radicals, e.g. organic nitroxo compounds, derivatives of oxazolidine, of piperidine or of pyrrolidine, which are known as so-called spin labels (see e.g. Chemie In Unserer Zeit 9, p. 18-24 and p. 43-49 (1975), Angew. Chem. 96, p. 171-246 (1984) and Chimia 40, p. 111-123 (1986).

For the provision of the compound according to the invention, at least one such detectable group is bound or coupled to the above described compound having aromatase inhibitory action. The binding or coupling may be covalent, ionic or a complexation. The binding or coupling of at least one detectable group should be carried out such that the inhibitory action towards the aromatase enzyme is not affected or diminished. However, it is easily possible to select those compounds which still have the inhibitory action towards the aromatase enzyme after the binding or coupling of the at least one detectable group. A corresponding selection is easily possible by means of a suitable method known in the prior art for the determination of the inhibition of the aromatase enzyme, such as the in-vitro-test described by E. A. Thompson and P. K. Siiteri in J. Biol. Chem. 249, p. 5364 ff. (1994).

Suitable processes for the modification or derivatisation of the above mentioned base compounds for the binding or coupling of the desired detectable group are well known to the person skilled in the art. Examples for basically suitable synthesis routes are the following, wherein it is possible to proceed analogous to the literature citations indicated in the parantheses:

Substitution reactions at the desired C-position of the steroid main skeleton as well as conjugations at the A-, B-, C- and/or D-rings of the steroid main skeleton via suitable Grignard or organometal reactions (see e.g. P. K.

Li and R. W. Brueggemeier in J. Med. Chem. 33(1), p. 101-105(1990));

Halogenisation at the desired positions at the basic steroid skeleton (see e.g. S. N. Perkins et al. in Carcinogenesis 18(5), p. 989-994 (1997));

Iodinisation of carbonyl groups at the basic steroid skeleton (see e.g. M. A. Hassan and B. M. Shabsoug in Acta Pharm. Hung. 67(6), p. 263-266 (1997));

Acetylation, thioacetylation and a corresponding coupling of metal-chelatising structural moieties at the basic steroid skeleton (see e.g. G. Pouskouleli and I. S. Butler in Steroids 44(2), o. 123-136 (1984)).

For the provision of the preferred compounds according to the invention, wherein the detectable group is defined by at least one radioactive iodine isotope and/or at least one metal isotope, the following procedure is of particular practical advantage. In the case of the radioactive iodination, the corresponding non-radioactive halogen derivative is provided first (wherein halogen means fluorine, chlorine, bromine or iodine), whereupon the radioactive iodine subsequently replaces the non-radioactive halogen by means of a suitable substitution reaction. In the case of using metal isotopes, several possibilities are taken into account. Firstly, a corresponding precursor compound is provided, which comprises a suitable activating group, wherein the activating group may be subsequently easily subjected to a substitution reaction with a radio-nuclide via an electrophilic substitution. Preferred activating groups include e.g. a tributyltin-, a trimethylsilyl-, a t-butyldimethylsilyl- and a iodine group. A second, well suitable alternative consists in binding a metal-chelating group, e.g. ethylenediamine tetraacetate (EDTA), diethylenetriamine pentaacetate (DTPA), diamine triacetates, 1,3-diketones, to the basic skeleton, and in replacing the previously used non-radioactive metal counter-ion, e.g. the sodium or potassium ion, by the then desired metal isotope through simple incubation steps. A further suitable method for the introduction of radioactive metals consists in reacting the basic compound with a metal-peroxosalt (e.g. sodium pertechnitate or sodium perrhenate) in the presence of a reducing agent having sufficient redox potential, e.g. tin chloride, in a suitable solution. The radio-nuclides may also form a clustering structural moiety.

The compound having inhibiting action towards the aromatase enzyme, which is provided in a further aspect of the invention, is characterized in that it is derivatised with iodine, boron and/or a metal-chelating group or with a residue containing one of the mentioned groups. The thus obtainable non-radioactive compound may excellently be utilized by being converted, as precursor compound, into the corresponding detectable compound of the invention in a simple manner. Thus, stable iodine may be replaced by radioactive iodine (e.g. ¹²³J, ¹²⁵J and/or ¹³¹J) by means of substitution, boron or the boron compound group may be converted in a radioactive α-emitter by means of neutron irradiation, and the metal-chelating group, which is bound in advance to a preferably mono-valant counter-cation, e.g. sodium, potassium, lithium, ammonium or the like, can be reacted with the desired radioactive metal isotope. The synthesis and optionally the modification of the basic compound having inhibitory action towards the aromatase may be carried out in the same or in an analogous manner as described above. Besides, the descriptions above with respect to the compound directly bound to the detectable group applies correspondingly.

The compounds according to the present invention described above are particularly suitable for the use in medical diagnosis of mammals, particularly for humans. With the compound of the invention, a disease-determined increased amount of the aromatase enzyme, or an enhanced activity thereof may be detected. In this connection the diagnosis of estrogen-dependent diseases and of tumor diseases are particularly meaningful. This particularly applies to disease conditions, above all to tumor diseases, of the breast, the prostate, the colon, the kidney, the melanoms, the gliomas, the neuroplastomas and the like.

The compound according to the invention however may, depending on the desire, generally be used for the detection of the aromatase being present in the body via the detectable group bound to the compound.

The diagnostic determination is carried out here in a suitable manner on the basis of an imaging method being adapted to the detectable group. In the case of using a radioisotope as detectable group, the target tissue being marked or labeled with the compound of the invention, e.g. an aromatase-containing tissue or tumor, may be localized and detected by means of a suitable beam detector, e.g. an α-beam detector. A suitable method for example is the scintigraphy, the γ-scanning and the use of a γ-camera. Tomographic imaging methods such as the computer-tomography, the single photon emission-computer-tomography (SPECT) and the positron-emission-tomography (PET) may be used for the improvement of visualization as well. Magnetically detectable groups and ESR-detectable groups are to be carried out also through corresponding, well known NMR- or ESR-spectroscopic observations or imaging methods.

A particularly useful application field of the compound of the invention having the detectable group consists in the medical therapy, wherein the diseases already mentioned above in connection with diagnostic analysis, particularly the tumor therapy, are contemplated. An advantageous combination of diagnosis and therapy may be utilized thereby easily. After the localization of the pathologic tissue according to the diagnostic possibilities described above, a therapeutic approach is enabled substantially more selective and more directed, e.g. in a conventional manner by means of local surgical operation. A particularly useful therapeutic approach, which is enabled by the provision of the compound of the invention, is carried out such that a therapeutically active radioisotope, e.g. ¹³¹J, ⁹⁰Y, ²¹¹At, ⁷⁵Br, ⁷⁷Br, ²¹²Pb, ²¹²Bi or the like, is used as the detectable group, in order to exhibit the radiotherapeutic action, while the portion of the compound of the invention, which provides for the aromatase-inhibitory action, concurrently achieves an additional therapeutic effect, causing a synergistic action.

The compound according to the invention may be administered to the patient to be tested or to be treated for medical diagnosis and/or medical therapy through a type of administration being known to the person skilled in the art and being appropriate depending on the application case. A variety of those types are known to the person skilled in the art, e.g. orally in the form of tablets, capsules, coated tablets, solutions or suspensions; rectally in the form of suppositories; parenterally, e.g. intra-muscular; or via intravenous injection or infusion; as well as topically via suitable types of formulation such as ointment, cream, gel, emulsion or lotio, powder, oil or the like. Hereby, the local topic application on the skin is particularly preferred, because in this manner a desired administration of the compound of the invention can be carried out substantially more selective and more directed in appropriate dosis. The compound according to the invention is normally used together with suitable vehicles and/or common additives, which fit to the respective type of application. Solid formulations contain e.g. diluents such as lactose, dextrose, saccharose, cellulose, starch as well as their derivatives; lubricants such as silica, talc, stearinic acid, magnesium or calcium stearate and/or polyethylene glycols; binders such as starch substances, gummi arabicum, gelatins, methylcellulose, carboxymethyl cellulose and other cellulose derivatives and/or polyvinyl pyrrolidone; disintegrating agents such as starch, alginic acid, alginates and/or sodium starch-glycolase; dyes; sweeteners; water-wetting agents such as lecithine, polysorbate, laurylsulfate; as well as generally non-toxic and pharmacologic inactive substances which are used in conventional pharmaceutical formulations. These pharmaceutical preparations may be prepared in known manner, e.g. by mixing, granulating, tabletting, coating or the like. Liquid dispersions for the oral administration may be syrups, emulsions and suspensions. Syrups may contain, as carrier, e.g. saccharose or saccharose in combination with glycerol and/or mannitol and/or sorbitol. Suspensions and emulsions may contain, as carrier, e.g. agar, sodium alginat, pectine, methylcellulose, carboxymethylcellulose or polyvinyl alcohol. Suspensions or solutions for intramuscular injections may contain, as carrier, e.g. sterile water, olive oil, ethyl oleate, glycols such as e.g. propylene glycol, and, if required, a small amount of local anaesthetic such as Lidocain. The solutions for intravenous injection or infusion may contain, as carrier, e.g. sterile water and particularly sterile, aqueous isotonic salt solutions. The suppositories may contain, as carrier, e.g. cacao butter; polyethylene glycol; a surface-active agent, e.g. polyoxyethylene/sorbitane/fatty acid ester or lecithine. The topical formulations may contain, as carrier, e.g. plant oils such as almond oil, olive oil, peanut oil; plant extracts; etheric oils; vitamin oils; fats and lipid-like substances, lipoids, phospholipids; carbohydrates such as paraffins, vaseline, lanolin, waxes and the like; detergents and other skin-active substances such as lecithine, wool grease alcohols, carotene, skin nutritients, perfume, cosmetic substances, alcohols, glycerol, glycols, urea, talc, preservative agents, sun screen agents, pigments such as titan-white and zinc-white, antioxidants, etc. Water generally serves as base substance, so that—typically by using emulsions such as fatty alcohol sulfates, alkali soaps, lecithines, triethanolamine and the like—an O/W- or W/O-emulsion is obtained.

The dosis of the compound of the invention may vary in broad ranges, depending on the demand and the application case, particularly in view of the respective type of application and whether a diagnostic and/or a therapeutic use is of primary importance, and usually lies in the range of from 0.01 mMol of the compound per kg bodyweight to about 500 mMol of the compound per kg bodyweight, preferably in the range of from 5 to 50 mMol of the compound per kg bodyweight. In the case of the diagnostic and/or therapeutic approach on the basis of radioactive labeled compounds, the amount of the administered radioactivity may be different depending on the type of radioisotope, and may vary e.g. in the range of from 10 MBq to 30,000 MBq per administered dosis, preferably in the range of from 300 to 20,000 MBq. The specific activity of the radioactive compound applied for the diagnostic imaging or for therapy is preferably rather high. For example, the specific radioactivity is more than 3·101⁰ Bq/mMol to 3·10¹⁵ Bq/mMol, preferably 3·10¹³ to 2·10¹⁵ Bq/mMol. The respective dosis or specific radioactivity however may optionally be adapted depending on the individual case.

1. A compound which exhibits an inhibitory action towards the aromatase enzyme and wherein the compound represents a steroidal aromatase inhibitor and comprises a detectable group.

2. The compound according to claim 1, wherein the compound has a molecular skeleton which is derived from androst-4-ene-, androst-5-ene-, androsta-1,4-diene- or androsta-1,4,6-triene- having a -3,17-dione-, a -6,17-dione-, a -7,17-dione-, a -3,6,17-trione- or a -4,7,17,19-tetraone- group, as well as the derivatives and analogs of the mentioned base structures.

3. The compound according to claim 1 wherein the compound exhibits an irreversible inhibitory action to the aromatase enzyme.

4. The compound according to claim 1, wherein the detectable group is a radioactive group.

5. The compound according to claim 4, wherein the radioactive group comprises at least one isotope which is selected from the group of radioactive iodine isotopes or of radioactive metal isotopes.

6. The compound according to claim 1, wherein the detectable group comprises a magnetically active isotope.

7. The compound according to claim 1, wherein the detectable group is a group detectable by ESR.

8. A compound which exhibits an inhibitory action to the aromatase enzyme and wherein the compound represents a steroidal aromatase inhibitor and is derivatized by iodine, boron and/or a metal-chelating group, or by a structural moiety containing iodine, boron and/or a metal-chelating group, except for the compound TAN-931 derivatized by iodine.

9. The compound according to claim 8, wherein the compound has a molecular skeleton which is derived from androst-4-ene-, androst-5-ene-, androsta-1,4-diene- or androsta-1,4,6-triene- having a -3,17-dione-, a -6,17-dione-, a -7,17-dione-, a -3,6,17-trione- or a -4,7,17,19-tetraone- group, as well as the derivatives and analogs of the mentioned base structures.

10. The compound according to claim 8 wherein the compound exhibits an irreversible inhibitory action to the aromatase enzyme.

11. A method for medical diagnosis comprising administering, to the patient to be tested for medical diagnosis, a compound according to claim 1 or a compound according to claim 8, and detecting whether a disease-determined increased amount of the aromatase enzyme, or an enhanced activity of the aromatase enzyme is present or not.

12. The ue method according to claim 11, wherein the compound is used for tumor diagnosis.

13. A method for the detection of aromatase Present in the body comprising administering to the patient to be tested a compound according to claim 1 or a compound according to claim 8, and detecting for the detection the presence of aromatase in the body via the detectable group bound to the compound.

14. A method for medical therapy comprising administering, to the patient to be treated for medical therapy, a therapeutically effective dose of a compound according to claim 1 or a compound according to claim 8.

15. The method according to claim 14, wherein the compound is used for tumor therapy.

16. The method according to claim 11, wherein a diagnostic determination is carried out on the basis of an imaging method being adapted to the detectable group.
